(19) United States
(12) Patent Application Publication
(10) Pub. No.: US 2024/0362787 A1
(43) Pub. Date: Oct. 31, 2024

(54) TUMOR CLASSIFICATION METHOD AND ANALYSIS DEVICE USING MULTI-WAVELENGTH PHOTOACOUSTIC IMAGE AND ULTRASOUND IMAGE

(71) Applicants: POSTECH RESEARCH AND BUSINESS DEVELOPMENT FOUNDATION, Pohang-si (KR); THE CATHOLIC UNIVERSITY OF KOREA INDUSTRY-ACADEMIC COOPERATION FOUNDATION, Seoul (KR)

(72) Inventors: Chulhong KIM, Pohang-si (KR); Jeonghoon HA, Seoul (KR); Byullee PARK, Suwon-si (KR); Dong Jun LIM, Seoul (KR)

(73) Assignees: POSTECH RESEARCH AND BUSINESS DEVELOPMENT FOUNDATION, Pohang-si (KR); THE CATHOLIC UNIVERSITY OF KOREA INDUSTRY-ACADEMIC COOPERATION FOUNDATION, Seoul (KR)

(21) Appl. No.: 18/771,155

(22) Filed: Jul. 12, 2024

Related U.S. Application Data
(63) Continuation of application No. PCT/KR2022/008020, filed on Jun. 7, 2022.

(30) Foreign Application Priority Data
Jan. 14, 2022 (KR) 10-2022-0005947

(51) Int. Cl. G06T 7/00; A61B 5/00

(52) U.S. Cl. CPC G06T 7/0014; A61B 5/0095; G06T 2207/10132; G06T 2207/30096

(57) ABSTRACT

A tumor classification method using multi-wavelength photoacoustic image and ultrasound image comprises the steps of: receiving, by an analysis device, an image set including photoacoustic frames and ultrasound frames collected over time for a predetermined period of time by each of a plurality of wavelength bands with respect to a subject; selecting, by the analysis device, photoacoustic frames and ultrasound frames to be analyzed, from the image set, on the basis of ultrasound image; performing, by the analysis device, spectral unmixing analysis on the selected photoacoustic frames; calculating, by the analysis device, at least one parameter for a tumor region by using the result of the spectral unmixing analysis; and classifying, by the analysis device, the subject's tumor by using the parameter.

### CROSS-REFERENCE TO RELATED APPLICATIONS

This application is a continuation of pending PCT International Application No. PCT/KR2022/008020 filed on Jun. 7, 2022, and which claims priority under 35 U.S.C 119 (a) to Korean Patent Application No. 10-2022-0005947 filed with the Korean Intellectual Property Office on Jan. 14, 2022. The disclosures of the above patent applications are incorporated herein by reference in their entirety.

### BACKGROUND

### 1. Technical Field

The following description relate to a technique for classifying a patient's tumor by using multi-wavelength photoacoustic images and ultrasound images.

### 2. Related Art

Imaging devices used to diagnose diseases include X-ray imaging (X-ray), computed tomography (CT), magnetic resonance imaging (MRI), nuclear medicine imaging, optical imaging, and ultrasound, etc. Various imaging devices have different strengths and weaknesses, so they often play complementary roles in diagnosing specific diseases, such as cancer diagnosis. Therefore, research on medical convergence imaging technology is actively underway to maximize the advantages by converging various medical imaging technologies. Photoacoustic imaging is a representative example of medical convergence imaging technologies that combine optical imaging and ultrasound imaging. Among them, multi-wavelength photoacoustic imaging refers to a technology that provides specific biometric information (e.g. hemoglobin, fat, oxygen saturation, etc.) by analyzing photoacoustic images obtained by using multiple laser wavelengths.

### SUMMARY

In one general aspect, there is provided a tumor classification method using multi-wavelength photoacoustic images and ultrasound images includes receiving, by an analysis device, image sets comprising photoacoustic frames and ultrasound frames collected over time for a predetermined period of time for a subject in each of multiple wavelength bands, selecting, by the analysis device, photoacoustic frames and ultrasound frames to be analyzed from the image sets on the basis of the ultrasound frames, performing, by the analysis device, a spectral unmixing analysis on the selected photoacoustic frames, calculating, by the analysis device, at least one parameter for a tumor region by using a result of the spectral unmixing analysis, and classifying, by the analysis device, a tumor of the subject by using the parameter.

In another aspect, there is provided an analysis device for classifying a tumor by using multi-wavelength photoacoustic images and ultrasound images includes an input device configured to receive image sets comprising photoacoustic frames and ultrasound frames collected over time for a predetermined period of time for a subject in each of multiple wavelength bands, a storage device configured to store a program that calculates parameters for a tumor region by analyzing multi-wavelength photoacoustic images, and a calculation device configured to select photoacoustic frames and ultrasound frames to be analyzed from the image sets on the basis of the ultrasound frames, and calculate at least one parameter for the tumor region by using a result of a spectral unmixing analysis on the selected photoacoustic frames.

### BRIEF DESCRIPTION OF DRAWINGS

FIG. 1 is an example of a tumor classification system using multi-wavelength photoacoustic images and ultrasound images.

FIG. 2 is an example of a process in which an analysis device classifies a tumor by using multi-wavelength photoacoustic images and ultrasound images.

FIG. 3 is an example of a process in which the analysis device calculates multiple parameters for a tumor region from multi-wavelength photoacoustic images and ultrasound images.

FIGS. 4A to 4C show the results of a statistical comparative analysis of parameters extracted from images between patients with malignant tumors and patients with benign tumors.

FIG. 5 is an example of results of multivariate classification using SVM.

FIGS. 6A to 6C show the results of analyzing multi-wavelength photoacoustic images of patients with malignant tumors and patients with benign tumors.

FIG. 7 shows the results of comprehensively using conventional ultrasound tumor evaluation results and tumor evaluation results using the above-described multi-wavelength photoacoustic images.

FIG. 8 is an example of the analysis device that classifies tumors by using multi-wavelength photoacoustic images and ultrasound images.

### DESCRIPTION OF EXAMPLE EMBODIMENTS

The technology described below may be variously modified and have several embodiments. Therefore, specific embodiments will be illustrated in the accompanying drawings and described in detail. However, it is to be understood that the technology described below is not limited to specific embodiments, but includes all modifications, equivalents, and substitutions included the scope and spirit of the technology described below. Terms such as “first,” “second,” “A,” “B,” and the like, may be used to describe various components, but the components are not limited by the terms, and are used only for distinguishing one component from other components. For example, a first component may be named a second component and the second component may also be named the first component without departing from the scope of the technology described below. The term and/or includes a combination of a plurality of related described items or any one of the plurality of related described items.

It should be understood that singular expressions include plural expressions unless the context clearly indicates otherwise, and it will be further understood that the terms “comprise” and “have” used in this specification specify the presence of stated features, steps, operations, components, parts, or a combination thereof, but do not preclude the presence or addition of one or more other features, steps, operations, components, parts, or a combination thereof.

Prior to the detailed description of the drawings, it is intended to clarify that the components in this specification are merely distinguished by the main functions of each component. That is, two or more components to be described below may be combined into one component, or one component may be divided into two or more components for more detailed functions. In addition, each of the constituent parts to be described below may additionally perform some or all of the functions of other constituent parts in addition to the main functions of the constituent parts, and some of the main functions of the constituent parts may be performed exclusively by other components.

In addition, in performing the method or the operation method, each of the processes constituting the method may occur in a different order from the specified order unless a specific order is explicitly described in the context. That is, each process may be performed in the same order as specified, performed at substantially the same time, or performed in the opposite order.

The technology described below is a technique for classifying tumors by using photoacoustic images and ultrasound images.

A photoacoustic signal is an acoustic signal generated in the process of thermal expansion that occurs when a biological tissue is irradiated with a laser and absorbs energy of the irradiated laser. A photoacoustic image is an image generated by applying a signal processing algorithm to a received acoustic signal. A biological tissue is composed of the combination of various types of molecules and is different in an absorption rate depending on the wavelength of a laser. Multi-wavelength photoacoustic images refer to images acquired by using lasers of various wavelengths.

Ultrasound imaging obtains images by transmitting pulse waves into the human body from tissues with differences in acoustic impedance and amplifying and converting reflected signals by a computer.

Hereinafter, a device that classifies tumors by using photoacoustic images and ultrasound images is referred to as an analysis device. The analysis device is a device that processes predetermined images and data. For example, the analysis device may be implemented as a device such as a PC, a smart device, or a server, etc.

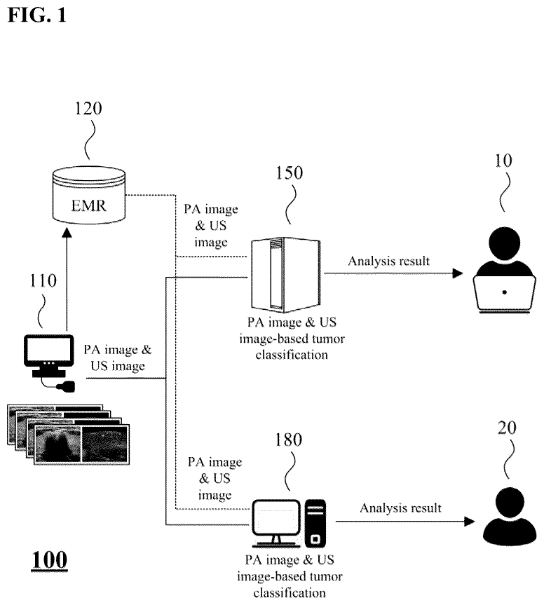

FIG. 1 is an example of a tumor classification system 100 using multi-wavelength photoacoustic images and ultrasound images.

The tumor classification system 100 may include an image generating device 110, an EMR 120, and analysis devices 150 and 180.

The image generating device 110 is a device that generates photoacoustic images (PA images) and ultrasound images (US images) for a subject. The subject is a person who wishes to receive diagnosis of a tumor condition (benign and malignant, etc.). The image generating device 110 may be a device that simultaneously generates a photoacoustic image and an ultrasound image. Alternatively, the image generating device 110 may be a device that includes each of photoacoustic images and ultrasound images. The image generating device 110 may generate photoacoustic images and ultrasound images for multiple wavelengths. The image generating device 110 may be a 3D imaging device.

Meanwhile, a researcher constructed a photoacoustic/ultrasound imaging system by combining a wavelength-convertible laser with a clinical ultrasound imaging system to obtain both photoacoustic images and ultrasound images. An imaging probe may include an ultrasonic sensor and an optical fiber in an adapter to acquire 2D photoacoustic/ultrasonic images. Photoacoustic images show light absorption characteristics within tissue, but does not show the structure of the tissue. However, ultrasound images show the structure in detail, and thus when ultrasound images are used, it is possible to easily specify which tissue a photoacoustic image signal is located at.

The image generating device 110 is able to transmit the photoacoustic images and ultrasound images of a subject to the EMR 120. The EMR 120 is able to store photoacoustic images and ultrasound images of patients.

FIG. 1 shows an analysis server 150 and an analysis PC 180 as examples of the analysis device. The analysis device may be implemented in various forms. For example, the analysis device may be implemented as a portable mobile device.

The analysis server 150 may receive the photoacoustic images and ultrasound images of a subject from the image generating device 110. The analysis server 150 may receive photoacoustic images and ultrasound images from the EMR 120. The analysis server 150 may classify the tumor of a subject by using the photoacoustic images and ultrasound images. An image processing process and a tumor classification process will be described later. The analysis server 150 transmits analysis results to a user 10. The user 10 may check the analysis results of the analysis server 150 through a user terminal. The user terminal refers to a device such as a PC, a smart device, and a mobile terminal, etc.

The analysis PC 180 may receive the photoacoustic images and ultrasound images of a subject from the image generating device 110. The analysis PC 180 may also receive photoacoustic images and ultrasound images from the EMR 120. The analysis PC 180 may classify the tumor of a subject by using photoacoustic images and ultrasound images. An image processing process and a tumor classification process will be described later. A user 20 may check analysis results through the analysis PC 180.

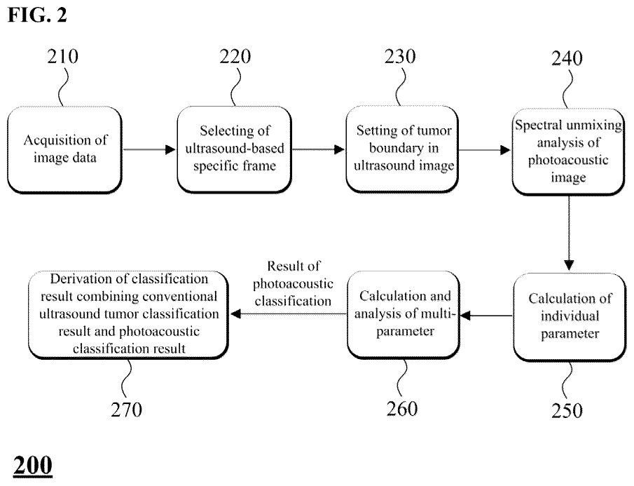

FIG. 2 is an example of a process 200 in which an analysis device classifies a tumor by using multi-wavelength photoacoustic images and ultrasound images. FIG. 2 is a schematic example of the process of classifying a tumor by using multi-wavelength photoacoustic images and ultrasound images.

The analysis device acquires continuous photoacoustic images of a subject and ultrasound images whose timing matches each photoacoustic image frame for multi-wavelength photoacoustic image analysis at 210. The photoacoustic images and ultrasound images may include continuous frames for the same point. Additionally, the photoacoustic images and ultrasound images may be composed of continuous frames that are generated with slightly different positions or directions over time.

The analysis device acquires multi-wavelength photoacoustic images and ultrasound images. In this case the value and number of wavelengths for the multi-wavelength photoacoustic images and the ultrasound images may be set in various ways. For convenience of explanation, it is assumed that multi-wavelength photoacoustic imaging and ultrasound imaging acquire M sets of photoacoustic/ultrasonic images including images for N wavelengths. Therefore, the total number of frames for each of the photoacoustic images and the ultrasound images is N*M.

Meanwhile, the quality of an image generated by the image generating device may vary depending on the movement of an operator (medical staff) or a patient. Accordingly, the analysis device may select a set of images with minimal shaking among multiple images. The analysis device may select a specific frame set to be analyzed on the basis of ultrasound images at 220. The analysis device may determine shaking information between frames on the basis of the ultrasound images. The analysis device arranges a total of N*M ultrasound frames in a row and calculates a correlation coefficient between ultrasound images included in an N-sized window. The analysis device moves one frame at a time in the window and calculates a total of N*M−(N−1) correlation coefficients. When the values of the correlation coefficients are high, frames within the corresponding window may be said to be images with little shaking. Accordingly, the analysis device may select top L sets with high correlation coefficient values among the total N*M−(N−1) sets of ultrasound images.

The analysis device may set tumor boundaries based on ultrasound images to perform tumor analysis on the selected image sets at 230. Meanwhile, the tumor boundary setting may be performed after a spectral mixing analysis, which will be described later.

The analysis device may set boundaries at a tumor location in the selected L sets of ultrasound images. The boundary setting may be performed by using a commercial tool or a self-developed tool. For example, the analysis device may set tumor boundaries that have characteristics different from surrounding normal tissue by using an image processing program. Alternatively, the analysis device may set tumor boundaries by using a deep learning model that segments a tumor region.

The analysis device determines a tumor region in photoacoustic images acquired at the same time as the corresponding ultrasound images by using information having the tumor boundaries set in the ultrasound images. Afterwards, the analysis device performs an analysis targeting the tumor region in the photoacoustic images.

The analysis device performs a spectral unmixing analysis on the photoacoustic images at 240. The spectral unmixing analysis may extract components such as hemoglobin (oxy-hemoglobin and deoxy-hemoglobin), melanin, and fat.

The analysis device may calculate individual parameters such as oxygen saturation on the basis of components obtained as the result of the spectral unmixing analysis at 250. For the individual parameters, various values may be used depending on the characteristics of a tumor (oxygen saturation, a distribution slope, and a photoacoustic slope, etc.).

The analysis device may perform multi-parameter analysis by combining the calculated individual parameters at 260. In this case, the analysis device may use a classification algorithm to classify benign and malignant. The analysis device may classify a tumor as benign or malignant by using a learning model. The learning model includes a decision tree, a random forest, a K-nearest neighbor (KNN), Naive Bayes, a support vector machine (SVM), and an artificial neural network (ANN). The analysis device may classify a tumor by using a specific pre-trained learning model.

Furthermore, the analysis device may additionally derive a final classification result by comprehensively using the results of the classification using the previous ultrasound images and the results of analyzing multiple parameters at step 260 (photoacoustic classification results) at 270. For example, the analysis device may combine the score of a tumor classification using only ultrasound images and a photoacoustic classification score calculated through 260 to ultimately derive a classification result as malignant or benign. Meanwhile, the score of the malignancy evaluation using the previous ultrasound images may be the result of evaluation by medical staff based on ultrasound images (TIRADS, thyroid cancer; BIRADS, breast cancer, etc.). Meanwhile, step 270 corresponds to an optional process.

The following description focuses on an experimental process in which a researcher analyzed actual images and classified tumors. The researcher classified tumors targeting thyroid cancer.

The analysis device may perform predetermined preprocessing before analyzing photoacoustic images and ultrasound images. For example, the analysis device may perform (i) the correction of deviations in acoustic resistance due to movement or the surrounding environment, (ii) the reconstruction of photoacoustic images by using a time delay beam forming (delay-and-sum method) algorithm, (iii) frequency demodulation for frequency domain detection, (iv) log compression for wide-range visualization, and (v) scanline transformation for image generation, etc.

At an affiliated medical institution, the researcher acquired (1) medical images (including biopsy results) taken the day before a surgery after hospitalization for patients scheduled to undergo total thyroidectomy, and (2) tumor classification results by performing fine-needle aspiration (FNA) examination for outpatients without biopsy results. Table 1 below shows information about a patient for whom a researcher obtained images.

TABLE 1

| | | | Operation | Size | Psammoma | | | | |
|---|---|---|---|---|---|---|---|---|---|
| No. | Type | Location | type | (mm) | body | TNM | Stage | BRAF | TERT |
| 1 | PTC | Left | T | 7 | N | T1aN0M0 | 1 | + | − |
| 2 | PTC | Right | L | 12 | Y | T1bN1aM0 | 1 | + | − |
| 3 | Benign | Left | N/A | | | | | | |
| 4 | PTC | Left | T | 12 | Y | T1bN1aM0 | 2 | + | − |
| 5 | PTC | Right | T | 26 | Y | T2N1bM0 | 1 | + | − |
| 6 | PTC | Left | T | 6 | N | T1aN1aM0 | 2 | − | − |
| 7 | PTC | Right | L | 8 | Y | T1aN1aM0 | 1 | + | − |
| 8 | Benign | Left | N/A | | | | | | |
| 9 | PTC | Right | N/A | | | | | | |
| 10 | PTC | Right | L | 6 | N | T1aN0M0 | 1 | + | N/A |
| 11 | PTC | Left | L | 10 | N | T1aN0M0 | 1 | + | N/A |
| 12 | PTC | Left | L | 6 | Y | T1aN1aM0 | 2 | + | N/A |
| 13 | PTC | Right | L | 6 | N | T1aN1aM0 | 1 | − | N/A |
| 14 | Benign | Left | N/A | | | | | | |
| 15 | Benign | Right | N/A | | | | | | |
| 16 | Benign | Right | N/A | | | | | | |
| 17 | PTC | Left | L | 12 | N | T1bN1aM0 | 1 | + | − |
| 18 | Benign | Left | N/A | | | | | | |
| 19 | PTC | Right | T | 11 | Y | T1aN0M0 | 1 | + | − |
| 20 | PTC | Right | T | 5 | Y | T1aN1aM0 | 2 | − | − |
| 21 | Benign | Right | N/A | | | | | | |
| 22 | PTC | Left | T | 7 | Y | T1aN0M0 | 1 | + | − |
| 23 | Benign | Left | N/A | | | | | | |
| 24 | PTC | Right | L | 6 | N | T1aN0M0 | 1 | + | − |
| 25 | PTC | Left | L | 15 | Y | T1aN1aM0 | 1 | + | − |
| 26 | PTC | Right | L | 10 | Y | T1aN1aM0 | 1 | + | − |
| 27 | Benign | Right | N/A | | | | | | − |
| 28 | PTC | Right | L | 6 | Y | T1aN1aM0 | 1 | − | |
| 29 | Benign | Right | N/A | | | | | | |
| 30 | Benign | Left | N/A | | | | | | |
| 31 | Benign | Left | N/A | | | | | | |
| 32 | Benign | Right | N/A | | | | | | |
| 33 | Benign | Left | N/A | | | | | | |
| 34 | Benign | Left | N/A | | | | | | |
| 35 | Benign | Right | N/A | | | | | | |
| 36 | Benign | Left | N/A | | | | | | |
| 37 | PTC | Left | L | 12 | N | T1bN0 | 1 | + | − |
| 38 | PTC | Right | T | 12 | Y | T1bN1b | 1 | + | − |
| 39 | Benign | Right | N/A | | | | | | |
| 40 | Benign | Right | N/A | | | | | | |
| 41 | Benign | Right | N/A | | | | | | |
| 42 | Benign | Left | N/A | | | | | | |
| 43 | Benign | Right | N/A | | | | | | |
| 44 | Benign | Right | N/A | | | | | | |
| 45 | Benign | Left | N/A | | | | | | |
| 46 | Benign | Right | N/A | | | | | | |
| 47 | Benign | Right | N/A | | | | | | |
| 48 | Benign | Left | N/A | | | | | | |
| 49 | PTC | Left | T | 16 | Y | T1bN1aM0 | 1 | + | − |
| 50 | Benign | Right | N/A | | | | | | |
| 51 | Benign | Left | N/A | | | | | | |
| 52 | PTC | Left | T | 9 | Y | T1aN1bM0 | 1 | + | − |

In Table 1, as for an operation type, T means total thyroidectomy, and L means lobectomy. N/A means that an operation type is unknown. TNM is information about tumors, crystals and metastases. BRAF and TERT are genetic test results, wherein ‘+’ indicates positive and ‘−’ indicates negative. The number on the left is the number of a patient, and in the type, PTC indicates a patient with a malignant tumor (papillary thyroid cancer, PTC), and Benign indicates a patient with a benign tumor.

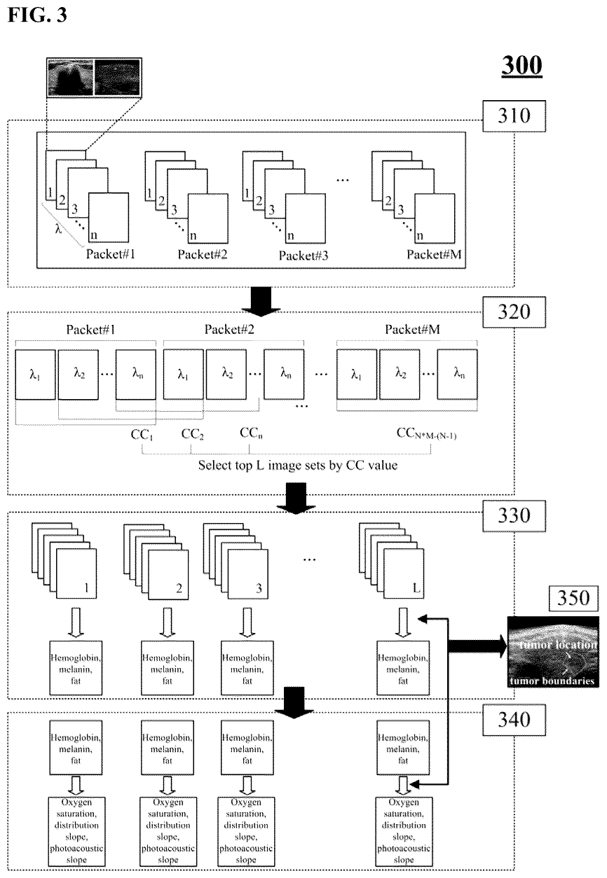

FIG. 3 is an example of a process 300 in which the analysis device calculates multiple parameters for a tumor region from multi-wavelength photoacoustic images and ultrasound images.

The analysis device receives multi-wavelength photoacoustic images and ultrasound images acquired for a predetermined period of time at 310. The analysis device acquires photoacoustic images and ultrasound images for n wavelength bands. A set of photoacoustic images and ultrasound images corresponding to one cycle for all wavelengths was defined as one packet. One packet includes n frames for photoacoustic images and ultrasound images, respectively. The analysis device processes data for M packets. Accordingly, all photoacoustic images and ultrasound images each acquired N*M frames. Meanwhile, the researcher acquired photoacoustic images and ultrasound images for each of five wavelengths. The five wavelengths were 700 nm, 756 nm, 796 nm, 866 nm, and 900 nm. Additionally, the researcher set one packet as 1 second of data, and used a total of 15 seconds (15 packets) of data.

The analysis device selects a specific frame among the acquired frames to improve accuracy at 320. The analysis device arranges a total of N*M ultrasound frames in a row and calculates a correlation coefficient (CC) between ultrasound images included in an N-sized window. Referring to FIG. 3, the correlation coefficient is calculated as a total of N*M−(N−1) correlation coefficients. The correlation coefficient CC may be calculated as in Equation 1 below.

$$C C ( [ {US}_{i} , {US}_{j} ) = \frac{cov ( {US}_{i} , {US}_{j} )}{{σ}_{i} {σ}_{j}} = \frac{\sum ( U {S}_{i} ( n ) - {μ}_{i} ) ( U {S}_{1} ( n ) - {μ}_{j} )}{{σ}_{𝔦} {σ}_{i}} \qquad [ Equation 1 ]$$

μᵢ and σᵢ respectively are the average and standard deviation of the pixel values of an ith ultrasound image, and μⱼ and σⱼ respectively are the average and standard deviation of the pixel values of a jth ultrasound image.

The analysis device selects L out of sets of N*M−(N−1) frames. The analysis device may select L at the top in order of CC values out of the sets of the N*M−(N−1) frames. At this time, the selected image sets may include both photoacoustic images and ultrasound images.

The analysis device performs the spectral unmixing analysis on the photoacoustic images among the selected L frame sets at 330. There may be several spectral unmixing methods in multi-wavelength photoacoustic imaging. A typical spectral unmixing method is to obtain a least-squares solution. The spectral unmixing technique enables spectral identification in multi-wavelength photoacoustic images obtained from a tumor and a surrounding tissue thereof. Therefore, the analysis device may distinguish between oxy-hemoglobin and deoxy-hemoglobin in blood through the spectral unmixing technique and calculate oxygen saturation through this. Additionally, the analysis device may distinguish tissues from images and classify components such as hemoglobin, melanin, and fat, etc.

The analysis device may calculate individual parameters within a tumor boundary (a tumor region) at 340. At this time, the individual parameters refer to parameters for each tumor region. Therefore, the analysis device is required to identify a tumor region in advance. To this end, the analysis device is required to identify a tumor region before or after spectral unmixing at 350. As described above, the analysis device may set tumor boundaries or detect a tumor region based on ultrasound images by using various image processing techniques or learning models. The analysis device detects the tumor region based on the ultrasound images, and may analyze the same region as the tumor region in photoacoustic images acquired at the same time.

The individual parameters may include various types of variable(s). For example, the analysis device may calculate at least one of the following individual parameters for the tumor region through the photoacoustic images.
(1) Calculation of oxygen saturation at a tumor region: the analysis device may calculate oxygen saturation by using oxy-hemoglobin and deoxy-hemoglobin obtained by the spectral unmixing technique.
(2) Calculation of the distribution of oxygen saturation in a tumor region: the analysis device may analyze the slope of the distribution by using the histogram distribution of oxygen saturation in the tumor region. The steeper the slope to the right, the higher the oxygen saturation.
(3) Calculation of a photoacoustic spectrum slope in a tumor region: the analysis device may calculate photoacoustic signals (the average value of the tumor region, etc.) at the tumor region of the photoacoustic images corresponding to n wavelengths, and may obtain a slope for the size of a photoacoustic signal for each wavelength.

Furthermore, the analysis device may calculate the amount of oxy-hemoglobin, deoxy-hemoglobin, and total hemoglobin, etc. for a tumor region as parameters.

The analysis device may calculate the aforementioned parameters by uniformly processing the photoacoustic signals for the tumor region. The analysis device may uniformly normalize initial photoacoustic signals to correct noise in the signals. The analysis device may determine linear regression for the photoacoustic signals by extracting the top 50% of the normalized signals and using first-order polynomial fitting for the average value thereof. In this case, the slope of the fitted line corresponds to a photoacoustic signal slope or photoacoustic slope.

The analysis device may calculate relative oxygen saturation (sO₂) for each pixel in a tumor region by using Equation 2 below.

$${s O}_{2} = \frac{{H b O}_{2}}{H bT} = \frac{{H b O}_{2}}{{H b O}_{2} + H bR} \qquad [ Equation 2 ]$$

HbO2 is an oxy-hemoglobin value, HbR is a deoxy-hemoglobin value, and HbT is a total hemoglobin value. Oxygen saturation for a tumor region may be calculated as the average value of oxygen saturation for the top 50% of the pixels of the tumor region. Additionally, the analysis device may quantify the pixel distribution of pixels of the top 50% of oxygen saturation in the tumor region. The analysis device may calculate a slope angle by connecting the center of a horizontal axis and a peak point in the Gaussian distribution of the oxygen saturation.

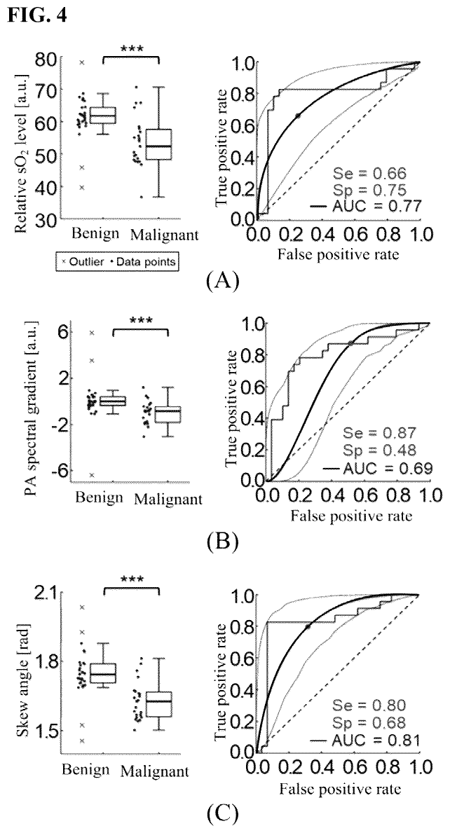

The researcher analyzed differences between patients with malignant tumors and patients with benign tumors in Table 1 on the basis of parameters extracted from the images (photoacoustic slope, oxygen saturation, and slope of oxygen saturation). FIGS. 4A-4C show the results of a statistical comparative analysis of parameters extracted from images between patients with malignant tumors and patients with benign tumors. FIG. 4A is the result of comparing oxygen saturation, FIG. 4B is the result of comparing photoacoustic slopes, and FIG. 4C is the result of comparing the slopes of the oxygen saturation distribution. Referring to FIG. 4, tumor classification based on oxygen saturation showed sensitivity (Se) of 66% and specificity (sp) of 75%. Tumor classification based on the slope of the photoacoustic signal for each wavelength showed sensitivity of 87% and specificity of 48%. Tumor classification based on the slope of the oxygen saturation distribution showed sensitivity of 80% and specificity of 68%. In FIG. 4, the bold solid line represents a receiver operating characteristic (ROC) curve. Referring to FIG. 4, it may be seen that AUC for each parameter distinguishing malignant from benign has a significant value. Therefore, it may be regarded that individual parameter(s) determined by the analysis device for a tumor region are effective in classifying the tumor of a corresponding subject.

Furthermore, the analysis device may classify tumors through multivariate analysis of multiple parameters for a tumor region. The analysis device may use a variety of multivariate classification techniques. The researcher classified tumors by using a support vector machine (SVM). The researcher used the scikit-learn C-Support vector classification algorithm of Python 3.6.5. The researcher used 80% of data prepared in advance as learning data and 20% thereof as verification data. SVM was trained to output a value of 1 for a benign tumor and −1 for a malignant tumor.

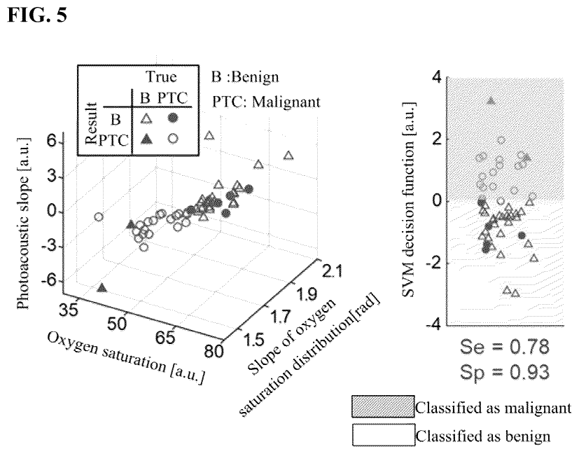

FIG. 5 is an example of results of multivariate classification using SVM. FIG. 5 shows the results of multi-parameter (photoacoustic slope, oxygen saturation, and the slope of oxygen saturation) analysis. Referring to FIG. 5, when SVM was used, sensitivity (Se) of 78% and specificity (Sp) of 93% were shown. Therefore, it may be seen that a multivariate classification method such as SVM is also effective in classifying tumors.

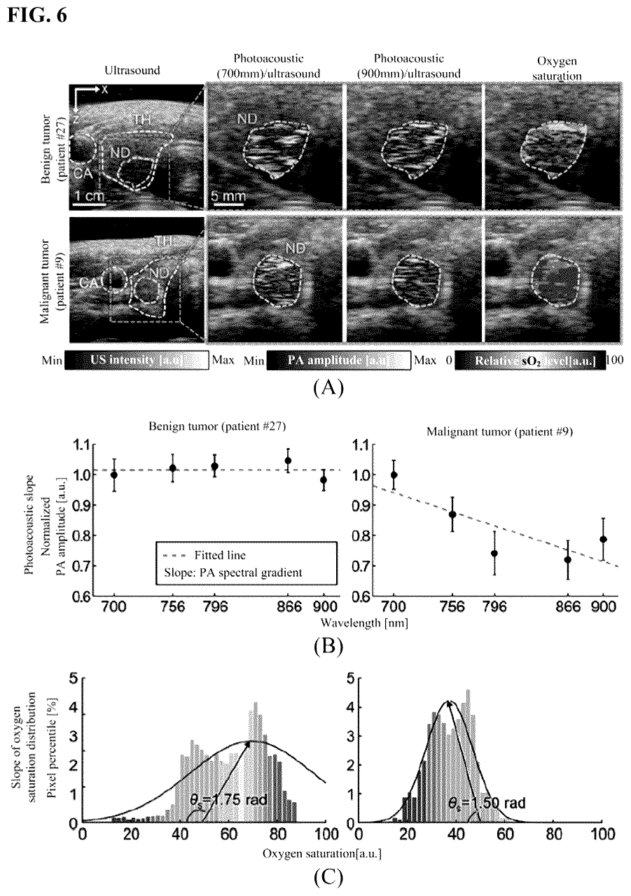

FIGS. 6A-6C show the results of analyzing multi-wavelength photoacoustic images of patients with malignant tumors and patients with benign tumors. FIG. 6 shows the results of analyzing multi-wavelength photoacoustic images in the method described above for images of a benign patient 27 and a malignant patient 9. FIG. 6A shows ultrasound images of benign and malignant thyroid tumors, photoacoustic images for wavelengths, and images of oxygen saturation which is an individual parameter. Referring to FIG. 6A, it may be seen that benign tumors and malignant tumors have different oxygen saturations. FIG. 6B shows a photoacoustic signal slope for each wavelength, which is one of individual parameters. Referring to FIG. 6B, the slope of patients with malignant tumors has a (−) slope, unlike that of patients with benign tumors. FIG. 6C is the slope of an oxygen saturation distribution, which is one of individual parameters. Referring to FIG. 6C, it may be seen that the distribution of patients with malignant tumors is skewed to the left, unlike that of patients with benign tumors.

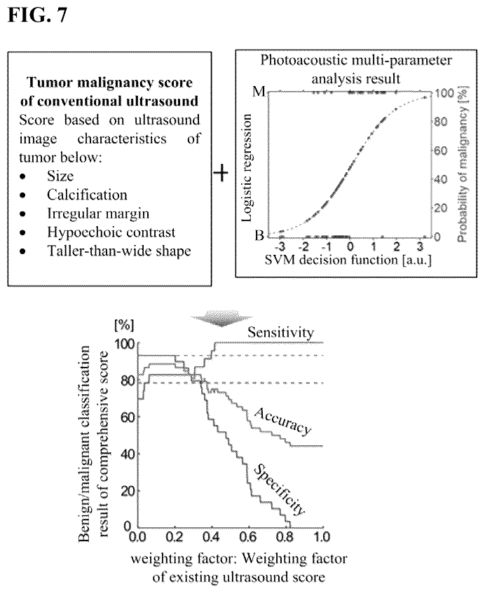

FIG. 7 shows the results of comprehensively using conventional ultrasound tumor evaluation results and tumor evaluation results using the above-described multi-wavelength photoacoustic images. FIG. 7 is an example using the results described in step 270 of FIG. 2. FIG. 7 is an example of combining the result of photoacoustic multi-parameter analysis with a conventional ultrasound image-based tumor score used in hospitals to improve a multi-parameter-based malignant/benign tumor classification performance. The conventional ultrasound image-based tumor score (a US guideline) is a result evaluated by a medical staff based on information that is able to be derived from an image. The ultrasound image-based tumor score varies depending on the region of a tumor, and typically, American thyroid association (ATA) and thyroid imaging reporting and data system (TI-RADS) are used for thyroid nodules, and a breast imaging reporting and data system (BI-RADS) is used for a breast cancer. The researcher evaluated a thyroid cancer by using an ultrasound image-based tumor score and a tumor evaluation score (multiple parameter analysis results in FIG. 3) for which the above-described multi-wavelength photoacoustic images are used. It was determined that an overall score=a*(a conventional ultrasound image-based tumor score)+(1−a)*(a multi-wavelength photoacoustic image-based multiple parameter analysis score). When a was set to be 0.2, sensitivity of 83% and specificity of 93% were observed. Meanwhile, when a was 0.41, sensitivity of 100% and specificity of 55% were shown. Therefore, it may be seen that the analysis device is effective in tumor classification when calculating a weighted sum with appropriate weights applied as a final overall score.

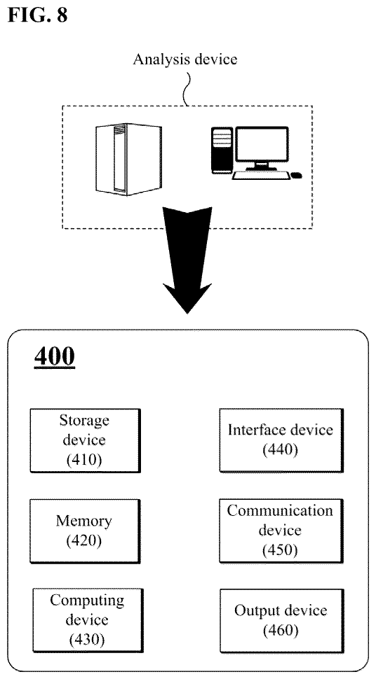

FIG. 8 is an example of the analysis device that classifies tumors by using multi-wavelength photoacoustic images and ultrasound images. The analysis device 400 corresponds to the analysis device (150 and 180 in FIG. 1) described above. The analysis device 400 may be implemented in physically various forms. For example, the analysis device 300 may take the form of a computer device such as a PC, a network server, or a chipset dedicated to data processing, etc.

The analysis device 400 may include a storage device 410, memory 420, a calculation device 430, an interface device 440, a communication device 450, and an output device 460.

The storage device 410 may store multi-wavelength photoacoustic images and ultrasound images of a subject.

The storage device 410 may store a program that uniformly preprocesses multi-wavelength photoacoustic images and ultrasound images.

The storage device 410 may store a program that calculates parameters for a tumor region by using multi-wavelength photoacoustic images and ultrasound images.

The storage device 410 may store scores for classifying a tumor in a conventional way (conventional ultrasound tumor evaluation results in FIG. 7) by using the ultrasound image of a subject.

The memory 420 may store data and information, etc. generated in a process in which the analysis device 400 classifies the tumor of a subject.

The interface device 440 is a device that receives certain commands and data from the outside. The interface device 440 may receive the multi-wavelength photoacoustic images and ultrasound images of a subject from an input device connected physically thereto or an external storage device. The interface device 440 may receive packets for multi-wavelength photoacoustic images and ultrasound images.

The communication device 450 refers to a component that receives and transmits certain information through a wired or wireless network. The communication device 450 may receive the multi-wavelength photoacoustic images and ultrasound images of a subject from an external object. The communication device 450 may receive packets for the multi-wavelength photoacoustic images and ultrasound images. The communication device 450 may transmit the analysis results of the subject to the external object.

The communication device 450 or the interface device 440 is a device that receives certain data or commands from the outside. The communication device 450 or the interface device 440 may be called an input device because the communication device 450 or the interface device 440 receives certain data.

A computing device 430 may consistently preprocess multi-wavelength photoacoustic images and ultrasound images of a subject.

The computing device 430 may select specific frames valid for analysis on the basis of the ultrasound images as illustrated in FIGS. 2 and 3.

The computing device 430 may set a tumor boundary or detect a tumor region on the basis of the ultrasound images in the selected frames. The computing device 430 may detect the tumor region in ultrasound images by using image processing techniques or learning models.

As illustrated in FIGS. 2 and 3, the computing device 430 may perform the spectral unmixing analysis on photoacoustic images. The computing device 430 may distinguish between hemoglobin, melanin, and fat components through the spectral unmixing analysis. The computing device 430 may calculate oxygen saturation by distinguishing between oxy-hemoglobin and deoxy-hemoglobin through the spectral unmixing analysis.

As illustrated in FIGS. 2 and 3, the computing device 430 may calculate individual parameters (a photoacoustic slope, oxygen saturation, the slope of oxygen saturation, etc.) for a tumor region in photoacoustic images. The computing device 430 may classify tumors on the basis of individual parameter(s) for the tumor region.

Furthermore, the computing device 430 may classify tumors by multivariate classification of the individual parameters. For example, the computing device 430 may classify tumors on the basis of multiple parameters by using a classification model such as SVM, etc.

Furthermore, as described at 270 of FIG. 2, the computing device 430 may perform final tumor classification by combining the results of conventional ultrasound-based tumor classification and the results of the tumor classification using parameter(s) based on multi-wavelength photoacoustic images.

The computing device 430 may be a device such as a processor, an AP, or a chip embedded with a program that processes data and performs predetermined calculations.

The output device 460 is a device that outputs certain information. The output device 460 may output interfaces and analysis results required for a data processing process. The output device 460 may output tumor classification results for a subject.

In addition, the medical image processing method or tumor classification method as described above may be implemented as a program (or application) including an executable algorithm that can be executed on a computer. The program may be stored and provided in a non-transitory computer readable medium.

The non-transitory computer-readable medium is not a medium that stores data for a short period of time, such as a register, a cache, a memory, or the like, but a medium that semi-permanently stores data and is readable by a device. Specifically, various applications or programs described above may be provided by being stored in non-transitory readable media such as a compact disc (CD), a digital video disc (DVD), a hard disk, a Blu-ray disc, a universal serial bus (USB), a memory card, a read-only memory (ROM), a programmable read only memory (PROM), an erasable PROM (EPROM), an electrically EPROM (EEPROM), or a flash memory.

The transitory readable media refer to various RAMs such as a static RAM (SRAM), a dynamic RAM (DRAM), a synchronous DRAM (SDRAM), a double data rate SDRAM (DDR SDRAM), an enhanced SDRAM (ESDRAM), a synclink DRAM (SLDRAM), and a direct rambus RAM (DRRAM).

The technology described above is a new approach to effectively analyze the phenotype of a sample using only gene expression information. The technology described above provides a diagnosis and treatment method for specific diseases by interpreting previously used gene ontology information into information that can be used to treat actual diseases.

The present embodiments and the drawings attached to the present specification merely clearly show some of the technical ideas included in the above-described technology, and therefore, it will be apparent that all modifications and specific embodiments that can be easily inferred by those skilled in the art within the scope of the technical spirit included in the specification and drawings of the above-described technology are included in the scope of the above-described technology.

What is claimed is:

1. A tumor classification method using multi-wavelength photoacoustic images and ultrasound images, the method comprising:
receiving, by an analysis device, image sets comprising photoacoustic frames and ultrasound frames collected over time for a predetermined period of time for a subject in each of multiple wavelength bands;
selecting, by the analysis device, photoacoustic frames and ultrasound frames to be analyzed from the image sets on the basis of the ultrasound frames;
performing, by the analysis device, a spectral unmixing analysis on the selected photoacoustic frames;
calculating, by the analysis device, at least one parameter for a tumor region by using a result of the spectral unmixing analysis; and
classifying, by the analysis device, a tumor of the subject by using the parameter.

2. The method of claim 1, wherein the analysis device arranges the ultrasound frames among the image sets according to wavelength band and time order, analyzes correlations between frames belonging to a corresponding unit in units of a predetermined number of frames, and selects units of a predetermined number of frames at a top in order of high correlation to select the photoacoustic frames and the ultrasound frames to be analyzed.

3. The method of claim 1, wherein the at least one parameter is at least one of oxygen saturation, an oxygen saturation slope, and a photoacoustic signal slope.

4. The method of claim 1, wherein the analysis device calculates multiple parameters for the tumor region and classifies the tumor of the subject by using a multivariate classification model for the multiple parameters.

5. The method of claim 3, wherein the multiple parameters include oxygen saturation, an oxygen saturation slope, and a photoacoustic signal slope.

6. The method of claim 1, wherein the analysis device finally classifies the tumor of the subject by combining a score of a conventional tumor classification using ultrasound images and a score of tumor classification using multiple parameters for the tumor region.

7. An analysis device for classifying a tumor by using multi-wavelength photoacoustic images and ultrasound images, the analysis device comprising:
an input device configured to receive image sets comprising photoacoustic frames and ultrasound frames collected over time for a predetermined period of time for a subject in each of multiple wavelength bands;
a storage device configured to store a program that calculates parameters for a tumor region by analyzing multi-wavelength photoacoustic images; and
a calculation device configured to select photoacoustic frames and ultrasound frames to be analyzed from the image sets on the basis of the ultrasound frames, and calculate at least one parameter for the tumor region by using a result of a spectral unmixing analysis on the selected photoacoustic frames.

8. The analysis device of claim 7, wherein the calculation device arranges the ultrasound frames among the image sets according to wavelength band and time order, analyzes correlations between frames belonging to a corresponding unit in units of a predetermined number of frames, and selects units of a predetermined number of frames at a top in order of high correlation to select the photoacoustic frames and the ultrasound frames to be analyzed.

9. The analysis device of claim 7, wherein the at least one parameter is at least one of oxygen saturation, an oxygen saturation slope, and a photoacoustic signal slope.

10. The analysis device of claim 7, wherein the calculation device calculates multiple parameters for the tumor region and classifies the tumor of the subject by using a multivariate classification model for the multiple parameters.

11. The analysis device of claim 10, wherein the multiple parameters include oxygen saturation, an oxygen saturation slope, and a photoacoustic signal slope.

12. The analysis device of claim 7, wherein the storage device further stores a score of a conventional tumor classification using ultrasound images of the subject, and the calculation device finally classifies the tumor of the subject by combining the score of the conventional tumor classification and a score of tumor classification using multiple parameters for the tumor region.
